(19) United States
(12) Patent Application Publication
(10) Pub. No.: US 2011/0277188 A1
(43) Pub. Date: Nov. 10, 2011

(54) Change in Plant Architecture

(76) Inventors: Villoo Morawala-Patell, Bangalore (IN); Mahesh Venkataramaiah, Bangalore (IN); Suhas Nimbalkar, Bangalore (IN); Manjula Ramakrishna, Bangalore (IN); Suresh Sadasivam, Bangalore (IN)

(21) Appl. No.: 13/054,445

(22) PCT Filed: Jul. 7, 2009
(86) PCT No.: PCT/IB2009/006227

§ 371 Date: Apr. 11, 2011

(30) Foreign Application Priority Data
Jul. 14, 2008 (IN) 1698/CHE/2008

(51) Int. Cl. A01H 5/00; C12N 15/82

(52) U.S. Cl. 800/287; 800/278; 800/293; 800/294; 800/298; 800/323

(57) ABSTRACT

The present invention relates to method for generating plants having altered architecture by introducing into plants, isolated nucleic acid molecules that can be used to produce transgenic plants characterized by altered plant architecture, carbon and nitrogen partitioning, enhanced biomass and or improved harvestable yield and to plants so generated and parts of these plants. More particularly, the present invention relates to a method for modifying a plant so as to produce a plant exhibiting an altered phenotype. Also provided are isolated nucleic sequence that encodes GAD polypeptide, vectors capable of expressing such nucleic acid molecules, host cells containing such vectors, and polypeptide encoded by such nucleic acids.

### FIELD OF THE INVENTION

The present invention relates generally to methods for generating plants having changed architecture and to plants so generated and parts of these plants. More particularly, the present invention relates to a method for modifying a plant so as to produce a plant exhibiting an altered phenotype. Plants and parts of plants, such as flowering and reproductive parts including seeds, also form part of the present invention. The ability to modify the phenotype of a plant may be useful for producing plants with more highly desired characteristics.

### BACKGROUND OF THE INVENTION

Manipulation of plant architecture has been one of the greatest mainstays of plant improvement—perhaps second only to the discoveries of the nutritional requirements of plants. With the advent of the ‘gene revolution’, there are countless new opportunities for selective modification of plant architecture

Plants are complex structures which can be described in many different ways depending on the requirements of the application, e.g. for bio-mechanics, hydraulic architecture or micrometeorology, or for simulation of plant growth. There is a general agreement that plants can be regarded as a collection of components having specific morphological characteristics, organized at several scales (White, 1979; Barthelemy, 1991). Plant architecture is a term applied to the organization of plant components in space, which can change with time. At a given time, plant architecture can be defined by topological and geometric information. Topology deals with the physical connections between plant components, while geometry includes the shape, size, orientation and spatial location of the components.

Plant architecture is defined as the three dimensional organization of the plant body. For the parts that are above ground, this includes the spatial arrangement of leaves and other photosynthetic organs and floral organs on stems and branching pattern. Plant architecture is even today the best means of identifying a plant species and has been the only criterion for systematic and taxonomic classification for a long time (Reinhardt and Kuhlemeier, 2002). Since the leaves collect solar energy and are surfaces for gas exchange, their arrangement in plant canopies is crucial for light interception and photosynthesis. Interception of light by the plants is dependent on the plant architecture. In both natural and agricultural systems, thus plant fitness and yield are affected by plant architecture. The plants to maximize canopy light interception have evolved different adaptive traits and plant architecture is one of the major adaptive traits. To maximize light interception the modified plant architecture include modification of size, shape, angle of leaf, plant height, branches and tillers. Some plants can modify their canopy architecture transiently to maximize light interception.

Plant architecture is a genetically controlled trait and therefore it s heritable, nevertheless environmental factors, both abiotic and biotic, can modify canopy architecture. Abiotic factors that affect canopy architecture include soil moisture content, nutrient availability, temperature and light. While biotic factors include herbivores, pathogens and competition with other plants.

Plant architecture is of major agronomic importance, strongly influencing the suitability of plants for cultivation and yield. In agriculture the yield improving plant architecture can increase potential of crops. Dwarf cultivars have been developed with modified canopy architectures capable of better light interception in different crops (Coyne, 1980). One of the greatest successes of the green revolution, which led to major increase in productivity was based on the modification of plant architecture where the selection of dwarf wheat varieties with short and sturdy stems helped the plants to resist damage from wind and rain resulting in higher yield (Peng et al., 1999).

Abiotic factors that can affect plant architecture include resources for plant growth such as soil moisture, temperature and light, under sufficient supply of these resources plants attain growth rate close to their genetic potential with maximum fitness and express typical architectures. However under scarce supply of these resources plants undergo physiological and growth changes leading to modified architecture for increasing their fitness.

### PRIOR ART

### Genes in Plant Architecture

Plants continuously form new leaves that are arranged in regular patterns this is called phyllotaxis. Inhibition of auxin transport, either by a mutation in the auxin transport protein PIN1 or by chemical inhibitors of auxin transport, specifically abolishes organ formation at the shoot apical meristem (SAM), whereas stem growth and meristem perpetuation are not affected resulting in the formation of pinelike stalks (Okada et al., 1991; Reinhardt et al., 2000). P-glycoproteins PGPs are plasma membrane anion transporters PGPs in Arabidopsis transport the hormone auxin, which controls cell elongation, plant shape, root branching and fruit development. pgp mutants examined thus far have reduced auxin transport and are dwarfs that have varying degrees of tropic responses (Murphy et al., 2000; Noh et al., 2001). AVP1, a pyrophosphate-driven proton pump, is important in the establishment and maintenance of auxin gradients required for root growth and development. Plants that overexpress AVP1 (AVP1OX) have greater shoot & root mass & surface area. AVP1 is highly conserved across the plant kingdom, with similar effects of overexpression being observed in Arabidopsis, tomato and rice (Gaxiola et al., 2001; Drozdowicz et al., 200).

TWD is an immunophilin-like protein with a putative plasma membrane GPI anchor. TWD interacts with many proteins within the plant, including PGPs. pgp1 pgp19 double mutants resemble twd mutants, indicating that TWD mediates interactions between PGPs and other proteins. twd mutants are dwarfs, and all anatomical features have hypemutation resulting in shorter plants with organs that twist, notably the stems, leaves, and flowers, resulting delightfully unusual looking plants (Kamphausen et al., 2002; Geisler et al., 2003).

Recent evidence has implicated Trehalose-6-Phospate synthase (TPS) genes as important modulators of plant development and inflorescence architecture. In one example, trehalose appears to modulate inflorescence branching in maize (Satoh-Nagasawa et al., 2006). Inflorescence branching in maize is controlled by the RAMOSA genes, and one of the genes (RAMOSA3) encodes a trehalose biosynthetic gene that functions through the regulation of the transcription factor RAMOSA1 (Satoh-Nagasawa et al., 2006). Chary et al., 2008 have provided evidence indicating that class II TPS gene functions in the control of cell morphology in addition to functioning as a broad modifier of whole plant developmental phenotypes. They identified a cell shape phenotype-1 (csp-1) mutant that has a dramatic cellular effect in the leaf epidermis, resulting in loss of pavement cell lobes. In addition, csp-1 was shown to impact the cell morphology of trichomes, resulting in an altered pattern of branching. The mutant shows a range of developmental defects that include reduced stature, altered stem branching, and pronounced leaf serrations.

### GABA Shunt

Gamma-Amino butyric acid (GABA) is a four-carbon non-protein amino acid conserved from bacteria to plants and vertebrates. GABA is a significant component of the free amino acid pool. GABA has an amino group on the g-carbon rather than on the a-carbon, and exists in an unbound form. It is highly soluble in water: structurally it is a flexible molecule that can assume several conformations in solution, including a cyclic structure that is similar to prolinel. GABA is zwitterionic (carries both a positive and negative charge) at physiological pH values (pK values of 4.03 and 10.56).

It was discovered in plants more than half a century ago, but interest in GABA shifted to animals when it was revealed that GABA occurs at high levels in the brain, playing a major role in neurotransmission. Thereafter, research on GABA in vertebrates focused mainly on its role as a signaling molecule, particularly in neurotransmission. In plants and in animals, GABA is mainly metabolized via a short pathway composed of three enzymes, called the GABA shunt because it bypasses two steps of the tricarboxylic acid (TCA) cycle. The pathway is composed of the cytosolic enzyme glutamate decarboxylase (GAD) and the mitochondrial enzymes GABA transaminase (GABA-T) and succinic semialdehyde dehydrogenase (SSADH). The regulation of this conserved metabolic pathway seems to have particular characteristics in plants.

The pathway that converts glutamate to succinate via GABA is called the GABA shunt. The first step of this shunt is the direct and irreversible a-decarboxylation of glutamate by glutamate decarboxylase (GAD, EC 4.1.1.15). In vitro GAD activity has been characterized in crude extracts from many plant species and tissues (Brown & Shelp, 1989). GAD is specific for L-glutamate, pyridoxal 5′-phosphate-dependent, inhibited by reagents known to react with sulfhydryl groups, possesses a calmodulin-binding domain, and exhibits a sharp acidic pH optimum of ˜5.8. GAD genes from Petunia (Baum et al., 1993), tomato (Gallego et al., 1995), tobacco (Yu & Oh, 1998) and Arabidopsis (Zik et al., 1998) have been identified. The second enzyme involved in the GABA shunt, GABA transaminase (GABA-T; EC 2.6.1.19), catalyzes the reversible conversion of GABA to succinic semialdehyde using either pyruvate or a-ketoglutarate as amino acceptors. In crude extracts, in vitro GABA-T activity appears to prefer pyruvate to a-ketoglutarate. However, distinct pyruvate-dependent and aketoglutarate-dependent activities are present in crude extracts of tobacco leaf, and these can be separated from each other by ion exchange chromatography (Van Cauwenberghe & Shelp). Both activities exhibit a broad pH optimum from 8 to 10. The Michaelis constants (Km) of a pyruvate-specific mitochondrial GABA-T from tobacco, purified ˜1000-fold, are 1.2 mM for GABA and 0.24 mM for pyruvate (Van Cauwenberghe & Shelp).

The last step of the GABA shunt is catalysed by succinic semialdehyde dehydrogenase (SSADH; EC 1.2.1.16), irreversibly oxidizing succinic semialdehyde to succinate. The partially purified plant enzyme has an alkaline pH optimum of ˜9; activity is up to 20-times greater with NAD than with NADP (Shelp et al., 1995).

Indeed, interest in the GABA shunt in plants emerged mainly from experimental observations that GABA is largely and rapidly produced in response to biotic and abiotic stresses. The GABA shunt has since been associated with various physiological responses, including the regulation of cytosolic pH, carbon fluxes into the TCA cycle, nitrogen metabolism, deterrence of insects, protection against oxidative stress, osmoregulation and signaling.

This is for the first time a method employing the glutamate decarboxylase gene to change the morphological architecture in plants has been demonstrated. Attempts have been made in this direction using genes like P-glycoproteins, auxin transporters, plant harmones and proton pumps. No attempt has been made till date to use genes involved in the GABA shunt pathway, specifically glutamate decarboxylase to change the architecture of the plants. Previous attempts directed at two glutamate decarboxylase genes from rice OsGAD1 and OsGAD2, which were introduced simultaneously into rice calli via Agrobacterium to establish transgenic cell lines. Regenerated rice plants had aberrant phenotypes such as dwarfism, etiolated leaves, and sterility (Akama & Takaiwa, 2007).

### OBJECTS OF THE INVENTION

The present invention relates of a method of changing the plant architecture (in both monocotyledons and dicotyledons) via Agrobacterium-mediated transformation with a glutamate decarboxylase gene. Further more the present invention relates to a method of plant modification to express genes, related to plant architecture and to the plants produced using this method.

Compositions and methods for altering the architecture of the plants by manipulation of GAD gene family in transgenic plants are provided.

The present invention provides nucleotides sequences of GAD gene. The nucleotide sequence and polypeptides of the invention include GAD gene, protein and functional fragments or variants thereof.

The methods of the invention comprise introducing into a plant a nucleotide sequence and expressing the corresponding polypeptide within the plant. The sequences of the invention can be used to alter plant architecture, carbon and nitrogen partitioning, enhanced biomass and or improved harvestable yield in plants. The methods of the invention find use in improving biomass and harvestable yield of the plants.

Additionally provided are transformed plants, plant tissues, plant cells, seeds, and leaves. Such transformed plants, tissues, cells, seeds, and leaves comprise stably incorporated in their genomes at least one copy of a nucleotide sequence of the invention.

One embodiment of the invention is a method for plant characteristics, the method comprising:

a. introducing into a plant cell a recombinant expression cassette comprising a nucleotide sequence whose expression, alone or in combination with additional polynucleotides, functions as an effector of nitrogen use efficiency within the plant;
b. culturing the plant cell under plant forming conditions to produce a plant; and,
c. inducing expression of the nucleotide sequence to alter the architecture of the plant.

### Sequence Listing

SEQ ID 1 shows the nucleic acid sequence of Oryza saliva glutamate decarboxylase gene. The start and stop codons are in italic.

SEQ ID 0.2 shows amino acid sequence of Oryza saliva glutamate decarboxylase gene. The asterisk denotes the stop codon.

### BRIEF DESCRIPTION OF ACCOMPANYING FIGURES

FIG. 1 shows the plant transformation vector harboring the glutamate decarboxylase encoding DNA sequence.

FIG. 2 shows the different stages in the transformation of tobacco leaves with GAD gene through Agrobacterium mediated gene transfer

FIG. 3 shows the PCR confirmation of the transformed and regenerated T0 seedlings of tobacco with GAD gene with different combination of primers—a) HygR-gene forward and reverse; b) Gene specific forward and reverse and c) Gene forward and Nos reverse primers

FIG. 4 shows the confirmation of the expression of the introduced gene (GAD) in T0 seedlings of tobacco with GAD gene analyzed using RT-PCR on cDNA as template with GAD gene specific forward and reverse primers.

FIG. 5 shows the comparison of leaf size in T0 GAD transgenic tobacco with the wild type plants and transgenic plants with a gene other than the GAD gene grown in green house.

FIG. 6 shows comparison of plant height between T1 Seedlings from GAD transgenics (D1A, E2 and H1), which were positive for Hygromycin and the wild type seedlings when grown in pots in the green house.

FIG. 7 shows comparison of internodal distance between T1 Seedlings from GAD transgenics (D1A, E2 and H1), which were positive for Hygromycin and the wild type seedlings when grown in pots in the green house.

FIG. 8 shows comparison of number of leaves between T1 Seedlings from GAD transgenics (D1A, E2 and H1), which were positive for Hygromycin and the wild type seedlings when grown in pots in the green house.

FIG. 9 shows comparison of stem girth or thickness between T1 Seedlings from GAD transgenics (D1A, E2 and H1), which were positive for Hygromycin and the wild type seedlings when grown in pots in the green house.

FIG. 10 shows comparison of leaf characters like a) leaf length; b) Leaf breadth and c) leaf area between T1 Seedlings from GAD transgenics (D1A, E2 and H1), which were positive for Hygromycin and the wild type seedlings when grown in pots in the green house.

FIG. 11 shows comparison of total biomass between T1 Seedlings from GAD transgenics (D1A, E2 and H1), which were positive for Hygromycin and the wild type seedlings when grown in pots in the green house.

FIG. 12 shows comparison of total grain yield between T1 Seedlings from GAD transgenics (D1A, E2 and H1), which were positive for Hygromycin and the wild type seedlings when grown in pots in the green house.

FIG. 13 shows comparison of seed boldness (weight of 100 seeds) between T1 Seedlings from GAD transgenics (D1A, E2 and H1), which were positive for Hygromycin and the wild type seedlings when grown in pots in the green house.

### DETAILED DESCRPTION OF THE INVENTION

The following detailed description of the invention is provided to aid those skilled in the art in practicing the present invention. Even so, the following detailed description of the invention should not be construed to unduly limit the present invention as modifications and variations in the embodiments discussed herein may be made by those of ordinary skill in the art without departing from the spirit or scope of the present invention.

This invention relates to a purified and isolated DNA sequence having characteristics of glutamate decarboxylase.

According to the present invention, the purified and isolated DNA sequence usually consists of a glutamate decarboxylase nucleotide sequence or a fragment thereof.

Included in the present invention are as well complementary sequences of the above-mentioned sequences or fragment, which can be produced by any means.

Encompassed by this present invention variants of the above mentioned sequences, that is nucleotide sequences that vary from the reference sequence by conservative nucleotide substitutions, whereby one or more nucleotides are substituted by another with same characteristics.

According to the present invention, the above mentioned nucleotide sequences could be located at both the 5′ and the 3′ ends of the sequence containing the promoter and the gene of interest in the expression vector.

Included in the present invention are the use of above mentioned sequences in altering the architecture of the plants produced thereof. “plant architecture” means that after introduction of DNA sequence under suitable conditions into a host plant, the sequence is capable of enhancing the leaf size, internodal distance, stem thickness, biomass and the harvestable yield in the plants as compared to control plants where the plants are not transfected with the said DNA sequence.

The following definitions are used in order to help in understanding the invention.

“Chromosome” is organized structure of DNA and proteins found inside the cell.

“Chromatin” is the complex of DNA and protein, found inside the nuclei of eukaryotic cells, which makes up the chromosome.

“DNA” or Deoxyribonucleic Acid, contain genetic informations. It is made up of different nucleotides.

A “gene” is a deoxyribonucleotide (DNA) sequence coding for a given mature protein. “gene” shall not include untranslated flanking regions such as RNA transcription initiation signals, polyadenylation addition sites, promoters or enhancers.

“Promoter” is a nucleic acid sequence that controls expression of a gene.

“Enhancer” referes to the sequence of gene that acts to initiate the transcription of the gene independent of the position or orientation of the gene.

The definition of “vector” herein refers to a DNA molecule into which foreign fragments of DNA may be inserted. Vectors, usually derived from plasmids, functions like a “molecular carrier”, which will carry fragments of DNA into a host cell.

“Plasmid” are small circles of DNA found in bacteria and some other organisms. Plasmids can replicate independently of the host cell chromosome.

“Transcription” refers the synthesis of RNA from a DNA template.

“Translation” means the synthesis of a polypeptide from messenger RNA.

“Orinetation” refers to the order of nucleotides in the DNA sequence.

“Gene amplification” refers to the repeated replication of a certain gene without proportional increase in the copy number of other genes.

“Transformation” means the introduction of a foreign genetic material (DNA) into plant cells by any means of trasnfer. Different method of transformation includes bombardment with gene gun (biolistic), electroporation, Agrobacterium mediated transformation etc.

“Transformed plant” refers to the plant in which the foreign DNA has been introduced into the said plant. This DNA will be a part of the host chromosome.

“Stable gene expression” means preparation of stable transformed plant that permanently express the gene of interest depends on the stable integration of plasmid into the host chromosome.

While the invention is broadly as defined above, it will be appreciated by those persons skilled in the art that it is not limited thereto and that it also includes embodiments of which the following description gives examples.

### Example 1

### Isolation and Purification of GAD Gene Nucleotide Sequence from Rice and Construction of Plant Transformation Vector

The GAD gene is cloned downstream of a 35S cauliflower mosaic virus promoter and terminated with a NOS terminator, all operably linked.

### Plant Materials

Oryza saliva (cv Rasi) was used for preparation of nucleic acids. After germination of the seeds, they were grown in hydroponic solution in a culture room. The seedlings were treated with 150 mM NaCl for 7-16 h.

### RNA Extraction and EST Library Construction

The RNA was extracted from the whole seedlings. An EST library of the salt stressed RASI cDNA was constructed. An EST showing identity to glutamate decarboxylase was identified from the EST library.

### Identification and Isolation of Genes in the GABA Shunt

GABA accumulates in higher plants following the onset of a variety of stresses such as acidification, oxygen deficiency, low temperature, heat shock, mechanical stimulation, pathogen attack, drought and salt stress. Glutamate decarboxylase, the gene in the GABA shunt has been isolated from the salt stressed library of O. sativa.

### Cloning of Glutamate Decarboxylase Gene

The Glutamate decarboxylase gene has been cloned into a cloning vector and also into plant transformation vectors (biolistic and binary) under a constitutive promoter. The cDNA encoding the complete coding sequence of glutamate decarboxylase gene was amplified from the indica rice (cv. RASI) cDNA using the following pairs of primers tagged with BglII and EcoRI restriction enzyme sites (underlined nucleotide sequences)

Forward: 5′-GCGGATCCATGGTGCTCTCCAAGGCCGTCTC-3′
Reverse: 5′-GCGAATTCCTAGCAGACGCCGTTGGTCCTCTTG-3′

Using the following PCR conditions 94° C. for 1 min; 94° C. for 30 sec; 75° C. for 3 min (cycled for five times); 94° C. for 30 sec; 68° C. for 3 min (cycled for 30 times) with a final extension of 68° C. for 7 min.

The amplified cDNA consists of 1479 base pairs of nucleotides and encodes for a mature glutamate decarboxylase enzyme.

The amplified fragment was cloned into pGEMT easy vector. The gene was restriction digested at BamHI and EcoRI sites and ligated into a biolistic vector pV1. This biolistic vector was excised at BglII and EcoRI restriction sites (BglII and BamHI enzymes are isoschizomers) to confirm the presence of the gene. The gene was also confirmed by sequencing. The resultant vector (pV1-GAD) has the GAD gene (1.479 kb) driven by 35S Cauliflower mosaic virus (35S CaMV) promoter and NOS terminator along with the ampicillin resistance gene as a selectable marker.

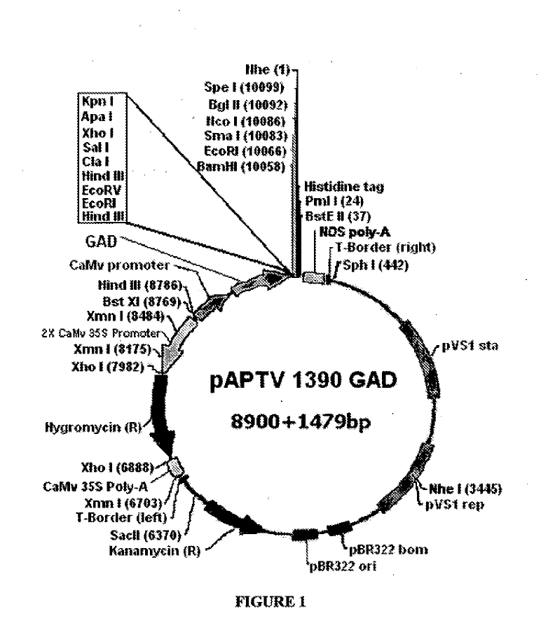

The gene cassette, GAD gene driven by the CaMV promoter and terminated by the NOS terminator from pV1-GD was restriction digested at HindIII and BamHI sites. This gene cassette was ligated into pCAMBIA 1390 pNG15 which was restriction digested at HindIII and BamHI sites. The resultant vector (pAPTV 1390-GAD) has the GAD gene (1.479 kb) driven by 35S cauliflower mosaic virus (35S CaMV) promoter and terminated by NOS terminator along with the nptII (Kanamycin resistance) gene and hph gene (Hygromycin resistance) as selectable markers (FIG. 1).

### Example 2

### Generating Plants with an Altered GAD Gene

### Plant Transformations

The Glutamate decarboxylase gene has been transformed via Agrobacterium into tobacco (model plant) and rice (crop plant) to arrive at the proof of concept for the identified gene.

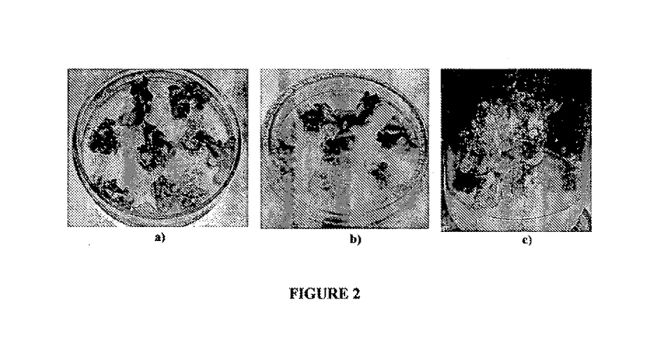

Detailed Steps Involved in Agrobacterium Mediated Transformation of Tobacco Leaf Explants with a Binary Vector Harboring Gad Gene:
1. The positive colony of Agrobacterium was inoculated in to LB broth with 50 mg/L Kanamycin (Kan) and 10 mg/L of Rifamicin (Rif) as vector backbone consists of Kan and Rif resistance gene, which also functions as double selection at one shot.
2. Then the broth was incubated at 28° C. on a shaker.
3. The overnight grown colony was inoculated into 50 mL LB broth with 50 mg/L Kan and 10 mg/L of Rif in the morning and incubated at 28° C. for 3-4 hours and the OD was checked at 600 nm and continued to grow till the OD was between 0.6-1.
4. Once the broth reached required OD the broth was centrifuged at 5000 rpm for 5 min.
5. The supernatant was discarded and the cell pellet was dissolved in Murashige & Skooge (MS) liquid medium (Agro-MS broth).
6. The tobacco leaves were cut in to small square pieces which served as explants with out taking the midrib and care was taken to injure leaf at all four sides with out injuring much at the center part of the inoculants.
7. These leaf samples were placed in MS Plain media for two days in a BOD incubator. After two days of inoculation these leaf samples were infected with transformed Agrobacterium cells, which are now in Agro-MS broth.
8. The leaf explants were placed in this Agro-MS broth for 30 min and then placed them on co-cultivation media, which consist of MS+1 mg/L 6-Benzyl amino purine hydrochloride (BAP)+0.2 mg/L Naphthalene acetic acid (NAA)+250 mg/L Cefotaxime for two days (FIG. 2a)
9. After co-cultivation the explants were kept in first selection medium which consist of MS+1 mg/L BAP+0.2 mg/L NAA+40 mg Hyg+250 mg/L Cefotaxime for 15 days and as the callus started protruding these explants were again sub cultured on to first selection media for callus to mature enough (FIG. 2b)
10. Once the callus was found to be matured these callus were inoculated on to second selection medium which consist of MS+1 mg/L BAP+0.2 mg/L NAA+50 mg Hyg+250 mg/L Cefotaxime. As the concentration of Hygromycin is increased the escapes from first selection get suppressed and only the transformed callus starts surviving on this media.
11. Subsequent sub-cultures on this second selection media were done once in ten days.
12. By this time the plantlets started protruding from the callus. The plantlets from second selection were taken and placed on to rooting media, which consist of ½ MS+0.2 mg/L Indole-3-butyric acid (IBA). Here the plantlets started protruding roots by 12-15 days. Once the mature roots were formed the plants were subcultured on to rooting media along with 20 mg/L of Hygromycin, as escapes can be identified at this stage also (FIG. 2c).
13. Plants at this stage were subjected to acclimatization where the caps of bottles were kept open for two days so that plants get adjusted to its growth room environment. Later plants from agar medium were removed and placed on ¼ MS liquid medium for two days. These plants were further transferred on to vermiculate and watered every day for one week.
14. Depending upon the condition of the plants suitable plants were transferred to green house.
15. Before sending plants to green house during acclimatization period old leaves from the plants were collected.
16. DNA from respective leaf samples was extracted and PCR with gene specific primers and selection marker gene i.e. Hygromycin primers were performed. PCR confirmed positive plants were further transferred to green house.
   Confirmation of Plants with Introduced Gad Gene

### Genomic DNA Extraction of GAD Tobacco Transgenic Lines

Leaf samples of transgenic GAD tobacco plant were collected and genomic. DNA was extracted.

### Procedure for Genomic DNA Extraction:

Around 1 gm of leaf was collected from each plant.
The samples were ground using liquid nitrogen in a pestle and mortar.
1 ml of extraction buffer Extraction buffer (0.2M Tris CI pH-8.0; 2 M NaCi; 0.05 M EDTA; 2% CTAB) was added to the sample and spun at 13K for 10 min
Supernatent was collected. RNase [3 μl (1 mg/mL) for 1 ml] was added and incubated at 37° C. for ½ an hour.
Equal volumes of chloroform-isoamyl alcohol was then added and spun at 13 k for 10 min.
Supernatant was collected in fresh tubes and equal volumes of chilled Isopropanol was added and spun at 13 k for 10 min.
The pellet was washed with 70% alcohol and pellet was dried and dissolved in 30 μl warm autoclaved water.
1 μl of DNA was loaded and checked on gel.
   The Transgenic Plants were Confirmed by PCR with Different Combination of Primers:

1. PCR with Hygromycin Forward (Hyg F) & Hygromycin Reverse (Hyg R) primers:

| Reagent | Stock | Volume | |
|---|---|---|---|
| Template DNA | | 1 | μl |
| Hyg F | 10 pM | 0.5 | μl |
| Hyg R | 10 pM | 0.5 | μl |
| dNTP's | 10 mM | 0.5 | μl |
| Taq DNA polymerase | 3 U/μl | 0.3 | μl |
| Taq buffer A | 10X | 3 | μl |
| Milli Q water | | 24.2 | μl |
| Total volume | | 30 | μl |

### PCR Conditions: (Eppendorf Machine)

| Steps | Temperature | Time | | Cycle |
|---|---|---|---|---|
| 1 | 94° C. | 3 | mins | |
| 2 | 94° C. | 30 | secs | |
| 3 | 50° C. | 50 | secs | |
| 4 | 72° C. | 1 | min | Go to step-2 30X |
| 5 | 72° C. | 10 | mins | |

| | | |
|---|---|---|
| 6 | 10° C. | ∞ |

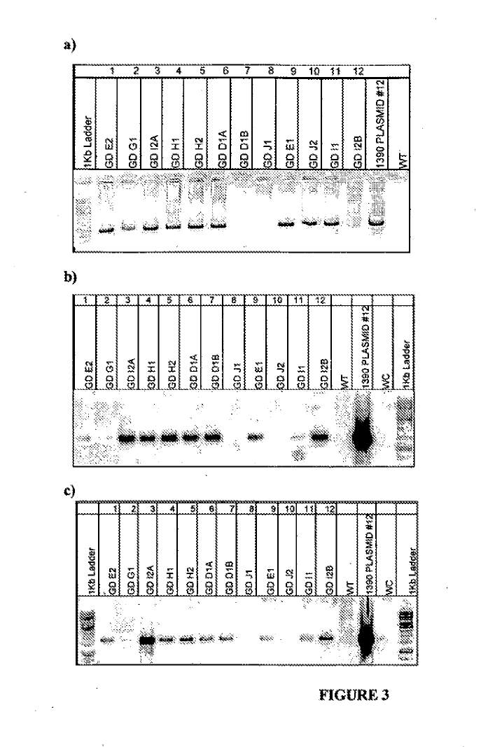

The amplified product was visualized on 0.8% agarose gel as shown in FIG. 3a.

2. PCR with Gene specific primers GAD Forward (GD F) & GAD Reverse (GD R):

| Reagent | Stock | Volume | |
|---|---|---|---|
| Template DNA | | 2 | μl |
| GD F | 10 pM | 0.5 | μl |
| GD R | 10 pM | 0.5 | μl |
| dNTP's | 10 mM | 0.5 | μl |
| Taq DNA polymerase | 3 U/μl | 0.3 | μl |
| Taq buffer A | 10X | 2 | μl |
| Milli Q water | | 14.2 | μl |
| Total volume | | 20 | μl |

### PCR Conditions: (Eppendorf Machine)

| Steps | Temperature | Time | | Cycle |
|---|---|---|---|---|
| 1 | 94° C. | 3 | mins | |
| 2 | 94° C. | 30 | secs | |
| 3 | 69° C. | 50 | secs | |
| 4 | 72° C. | 1.30 | min | Go to step-2 35X |
| 5 | 72° C. | 10 | mins | |

| | | |
|---|---|---|
| 6 | 10° C. | ∞ |

The amplified product was visualized on 0.8% agarose gel as shown in FIG. 3b.

3. PCR with GD F & Nos MR:

| Reagent | Stock | Volume | |
|---|---|---|---|
| Template DNA | | 2 | μl |
| GD F | 10 pM | 0.5 | μl |
| Nos MR | 10 pM | 0.5 | μl |
| dNTP's | 10 mM | 0.5 | μl |
| Taq DNA polymerase | 3 U/μl | 0.3 | μl |
| Taq buffer A | 10X | 2 | μl |
| Milli Q water | | 14.2 | μl |
| Total volume | | 20 | μl |

### PCR Conditions: (Eppendorf Machine)

| Steps | Temperature | Time | | Cycle |
|---|---|---|---|---|
| 1 | 94° C. | 3 | mins | |
| 2 | 94° C. | 30 | secs | |
| 3 | 67° C. | 50 | secs | |
| 4 | 72° C. | 2 | min | Go to step-2 35X |
| 5 | 72° C. | 10 | mins | |

| | | |
|---|---|---|
| 6 | 10° C. | ∞ |

The amplified product was visualized on 0.8% agarose gel as shown in FIG. 3c.

Primer sequences used in different PCR reactions are listed below:

Hyg F: 5′-CTGAACTCACCGCGACGTCT-3′
Hyg R: 5′-CCACTATCGGCGAGTACTTC-3′
GD F: 5′-GCGGATCCATGGTGCTCTCCAAGGCCGTCTC-3′
GD R: 5′-GCGAATTCCTAGCAGACGCCGTTGGTCCTCTTG-3′
NOS MR: 5′-GATAATCATCGCAAGACCGGCAAC-3′
Tub F: 5′-GACGAGCACGGCGTTGATCCTA-3′
Tub R: 5′-CCTCCTCTTCATACTCTTCCT-3′

### Confirmation of Expression of the Introduced GAD Gene in the Transgenic Plants

The confirmation of the expression of the introduced GAD gene involved steps like RNA extraction, cDNA synthesis and Reverse Transcription PCR.

RNA of transgenic GAD tobacco plants along with the control plant (wild type) was isolated.

### Detailed Steps Involved in RNA Extraction:

1. 500 mg of leaf tissue was taken in prechilled mortar and ground in liquid nitrogen to fine powder.
2. The powder was transfered to a prechilled eppendorf tube using a chilled spatula.
3. 1 ml of Trizol solution was added to the homogenized sample. Mixed well and incubated at room temperature (RT) for 5 min.
4. 200 μl of chloroform was added to it and shaken vigorously for 15 seconds and incubated at room temperature for 5 mins.
5. The samples were centrifuged at 13000 rpm for 15 min at 4° C.
6: The upper aqueous phase was collected in a fresh tube (Approximately 60% i.e. 600 μl)
7. 500 μl of cold Isopropanol was added to the upper phase collected and incubated at RT for 10 min.
8. The samples were centrifuged at 13000 rpm for 15 min at 4° C.
9. The supernatant was decanted and the pellet washed with 500 ul of 70% alcohol (DEPC H₂O) and centrifuged at 10000 rpm for 5 minutes at 4° C.
10. The supernatant was decanted and the pellet dried for 15 min at RT.
11. the pellet was dissolved in 20 μl of DEPC treated H₂O in a heating water bath or dry bath set at 55° C.
12. 2 μl of the sample is loaded on the gel. Stored the sample at −80° C. till further use.
   Detailed Steps Involved in cDNA Synthesis:

cDNA synthesis of transgenic GAD tobacco plants along with the wild type was done.
1. The components were added in the order given below:

Total RNA: 4 ul (1 ug)

Oligo dT's: 0.5 ul

0.1% DEPC/nuclease free water: 6.5 ul

Total: 11 ul
2. The contents heated at 70° C. for 5 min in a PCR machine and snap chill it in ice.
3. Meanwhile the next mixture was prepared by adding the following components in another tube:

5× reaction buffer: 4 ul

dNTP's (10 mM): 2 ul

RNase inhibitor (20 U/ul): 0.5 ul

0.1% DEPC/nuclease free water: 2 ul

Total: 8.5 ul
4. This 8.5 ul mixture was added to the content in PCR tube snap chilled and mix by gentle tapping.
5. The contents were incubated in PCR tube at 37° C. for 5 minutes in a PCR machine.
6. 0.5 ul of the M-MuLV RT enzyme was added to the tube and continued the program set in the PCR machine (25° C. for 10 min; 37° C. for 60 mM and 70° C. for 10 mM).
7. Store the cDNA at −20° C. till further use in PCRs.

### Analysis of Expression of the Introduced GAD Gene in the Transgenic Tobacco Plants by RT-PCR

The cDNA samples from GAD transgenic tobacco and wild type plant were analyzed by PCR with Gene specific primers to check for the expression of the introduced GAD gene in tobacco:

PCR of cDNA with Gene Specific Primers:

| Reagent | Stock | Volume | |
|---|---|---|---|
| Template cDNA (1:10) | | 2 | μl |
| GD F | 10 pM | 0.5 | μl |
| GD R | 10 pM | 0.5 | μl |
| dNTP's | 10 mM | 0.5 | μl |
| Taq DNA polymerase | 3 U/μl | 0.3 | μl |
| Taq buffer A | 10X | 3 | μl |
| Milli Q water | | 24.2 | μl |
| Total volume | | 30 | μl |

PCR Conditions (Eppendorf Machine):

| Steps | Temperature | Time | | Cycle |
|---|---|---|---|---|
| 1 | 94° C. | 3 | mins | |
| 2 | 94° C. | 30 | secs | |
| 3 | 69° C. | 50 | secs | |
| 4 | 72° C. | 1.30 | min | Go to step-2 30X |
| 5 | 72° C. | 10 | mins | |

| | | |
|---|---|---|
| 6 | 10° C. | ∞ |

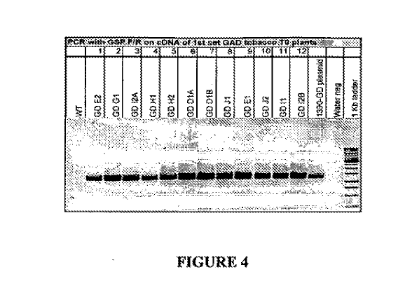

The amplified product was visualized on 0.8% agarose gel as shown in FIG. 4.

### Example 3

Evidence that Plants with Altered GAD Gene have an Altered Plant Architecture in to Generation

### Plant Material

The experiments were performed with the wild type and T0 transgenic tobacco plants. Seedlings were cultivated in a green house in pots containing mixture of field soil. Plants were irrigated with normal water, without any external application of fertilizers. The FYM mixed in the soil served as the only source of nutrition to the plants.

### Leaf Size

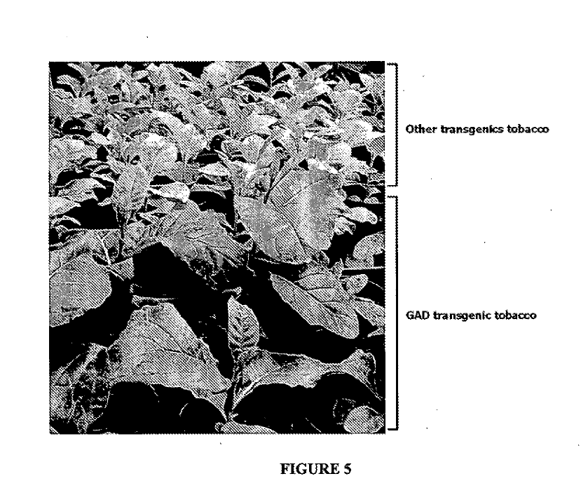

The size of the leaf was measured in the transgenic plants and the wild type plants (plants with out the introduced glutamate decarboxylase gene). The leaf size of the T0 transgenic plants was larger when compared with the leaves of the control plants. The leaf size increased at least 20% more than the control while the highest increase in leaf size was 160% over the wild type plants (Table 1 and FIG. 5)

TABLE 1
Comparison of leaf size between wild type
and T0 GAD tobacco transgenic plants

| Sl. No. | Plant | Leaf Diameter (Cms) | % Increase over control |
|---|---|---|---|
| 1 | D-1 | 19.5 | 95 |
| 2 | E-1 | 13 | 30 |
| 3 | E-2 | 12 | 20 |
| 4 | G-1 | 17.5 | 75 |
| 5 | H-1 | 12 | 20 |
| 6 | H-2 | 12.5 | 25 |
| 7 | I-1 | 18 | 80 |
| 8 | I-2 | 14 | 40 |
| 9 | J-1 | 10 | — |
| 10 | J-2 | 26 | 160 |
| 11 | Wild type | 10 | — |

### Example 4

### Evidence that Plants with Altered GAD Gene have an Altered Plant Architecture in T1 Generation

Phenotype of the transgenic plants was studied in the T1 generation to evaluate the changes in the plant architecture during the adult plant stage encompassing the whole life cycle of the plant.

The three transgenic event D1A, E2 and H1 were selected for evaluating the change in plant architecture in pot culture in the green house. The experiments were performed with the wild type and transgenic tobacco. The T1 seeds were germinated on moist filter paper discs supplemented with hygromycin (50 mg/L); the positive seedlings that germinated and grew on this were selected and placed on soil in big pots (11 inch diameter) along with the wild type seedlings. Seedlings were cultivated in a green house in pots containing mixture of field soil and farmyard manure (FYM). Plants were irrigated with normal water or saline water 200 mM NaCl. The experiments were performed with three replications with four genotypes (wild type and D1A, E2 and H1 transgenic tobacco) as indicated in Table 1.

TABLE 1
Experimental design for evaluation of change in plant architecture.

| | Genotypes | | | |
|---|---|---|---|---|
| Replication-1 | WT | D1A | E2 | H1 |
| Replication-2 | WT | D1A | E2 | H1 |
| Replication-3 | WT | D1A | E2 | H1 |

Three replications and four genotypes were taken for comparison.

### Phenotypic Evaluation:

The phenotypic characters were observed and parameters contributing plant architecture like plant height, internodal distance, number of branches, number of leaves, leaf area, stem thickness (girth), total biomass, grain yield etc were recorded.

### Plant Height

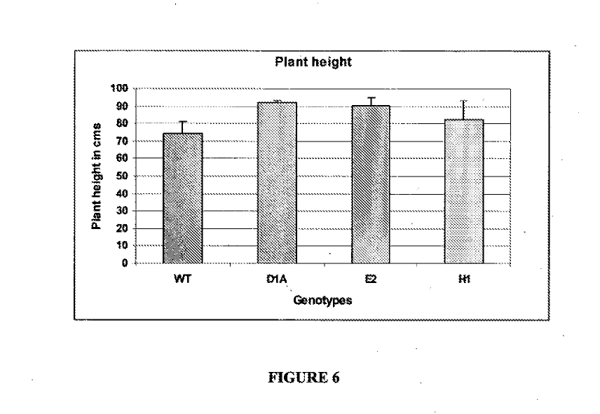

The height of the plant was measured in the transgenic plants and the wild type plants (plants with out the introduced glutamate decarboxylase gene). The plant height was measured using scale from the soil level to the tip of the plant including the inflorescence and the branches. The transgenic plants from the three events showed higher plant height as compared to the wild type plants (FIG. 6). There was at least 10% increase in the plant height (H1) and up to 23% increase in plant height (D1A) was observed

### Internodal Distance

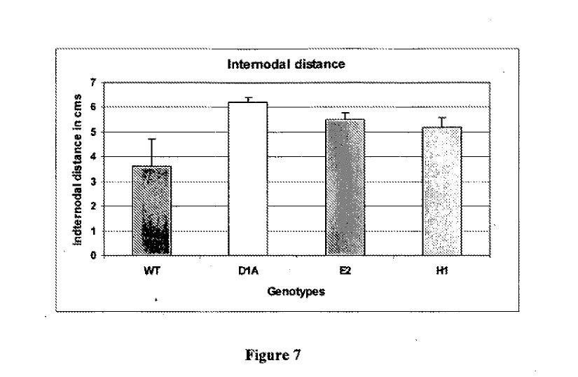

The distance between two internodes on the stem was measured in the transgenic plants and the wild type plants (plants with out the introduced glutamate decarboxylase gene). The internodal distance was measured between the 5^{th} & 6^{th} leaf and 6^{th} & 7^{th} leaf. The leaf was counted from the top with the fully expanded leaf considered to be leaf number-1. The distance was measured using a thread and then measuring the thread length on a scale and expressed in cms. The transgenic plant (H1) showed at least 44% increase in internodal distance as compared to the wild type (FIG. 7).

### Number of Leaves

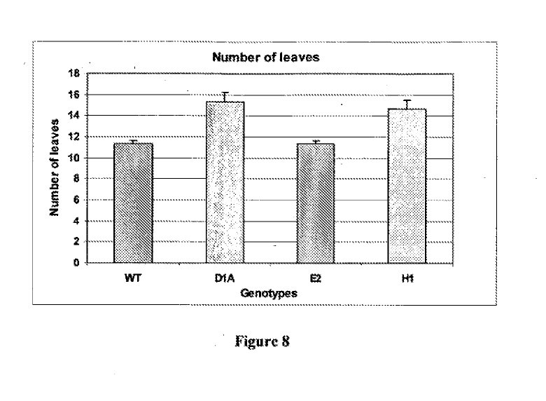

The number of leaves on each plant was counted in the transgenic plants and the wild type plants (plants with out the introduced glutamate decarboxylase gene). The transgenics exhibited 35% higher number of leaves when compared to wild type (FIG. 8).

### Stem Girth (Circumference or Stem Thickness)

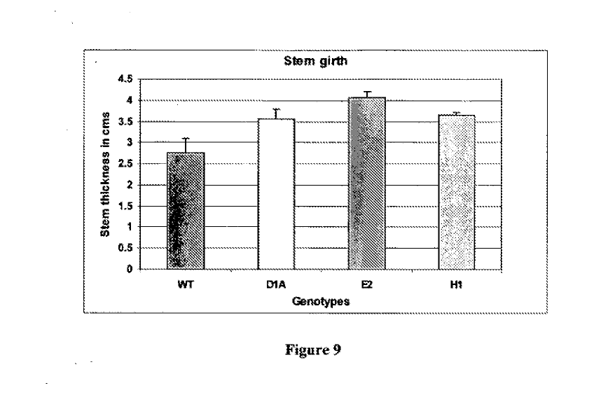

The thickness of the stem was measured in the transgenic plants and the wild type plants (plants with out the introduced glutamate decarboxylase gene). Girth of the stem was measured at a height of 5-6 cms above from the soil level. A thread was used to circle the stem at the appropriate height and then the length of the thread was measured on a scale and expressed in cms. The transgenics definitely had a thicker stem when compared to the wild type plants (FIG. 9). There was at least 28% thicker stems in the transgenics, however the stem thickness could be increased up to 47% (E2).

### Leaf Area

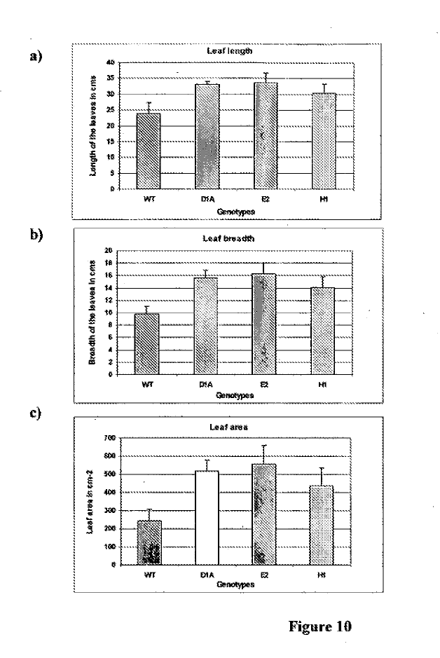

The size of the leaf was measured in the transgenic plants and the wild type plants (plants with out the introduced glutamate decarboxylase gene). The leaf was measured vertically from the node to the tip of the leaf and was considered as the length of the leaf. The transgenic plants possessed 27%-37% longer leaves than the wild type plants (FIG. 10a). The breadth of the leaf was measured horizontally at the broadest point and was considered as the breadth of the leaf. The transgenic plants exhibited 42%-65% more broader leaves than the wild type plants (FIG. 10b). The leaf area was calculated as the Length×Breadth expressed in cm⁻² units. There was significant increase (80%-129%) in the leaf area of the transgenics when compared to the wild type (FIG. 10c). The increase in leaf area has been stable over two generations tested (T0 and T1).

### Plant Biomass

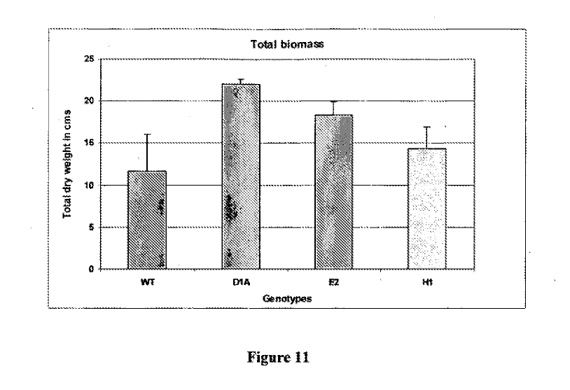

The biomass generated was measured in the transgenic plants and the wild type plants (plants with out the introduced glutamate decarboxylase gene). Plant biomass was estimated as the total plant dry weight. The total biomass from the transgenics was significantly higher (22%-88%) as compared to the wild types (FIG. 11). This could be due to the obvious fact that there is increase in other phenotypic characters like leaf size, stem thickness etc.

### Grain Yield

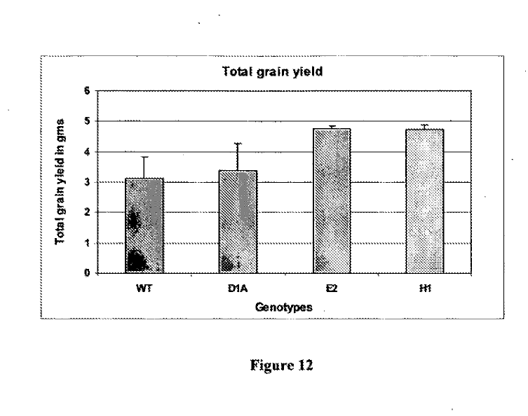

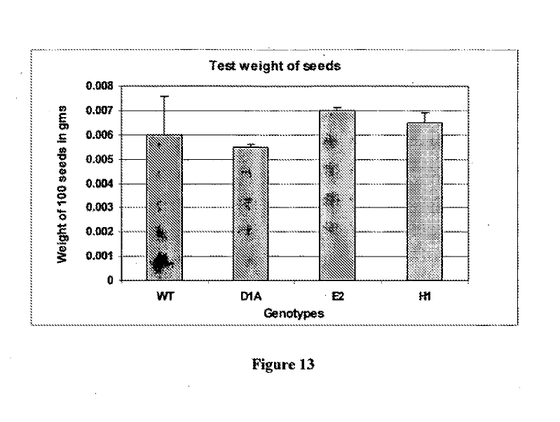

The total grain yield was significantly higher (up to 50% more) in the transgenics than the wild type (FIG. 12). The grains or seeds from the transgenic plants were also bolder or larger in size, which is indicated by the higher test weight of the seeds (FIG. 13).

To summarize the GAD transgenic plants from all the three events tested showed a positive altered phenotype or plant architecture. The GAD transgenic plants performed better than the wild type plants for the different agronomic and physiological status of the plants thus indicating the role of GAD gene for the altered plant architecture contributing towards the superior performance of the transgenic plants.

We claim:

1. A method for generating a transformed plant that exhibits altered plant architecture, comprising: incorporating into a plant's genome a DNA construct comprising a constitutive or non-constitutive promoter operably linked to a nucleotide sequence that encodes a functional glutamate decarboxylase (GAD) enzyme.

2. The method according to claim 1, wherein the nucleotide sequence that encodes a functional glutamate decarboxylase enzyme comprises a nucleotide sequence set forth in SEQ ID No. 1.

3. The method according to claim 1, wherein the promoter is selected from the group consisting of a constitutive promoter, an inducible promoter, a tissue specific promoter and a cell type specific promoter operably linked to the nucleotide sequence set forth in SEQ ID No. 1.

4. The method according to claim 3, wherein the promoter selected is from an inducible promoter, responds to a signal selected from the group consisting of mechanical shock, heat, cold, salt, flooding, drought, wounding, anoxia, pathogens, ultraviolet-B, nutritional deprivation, a flowering signal, a fruiting signal, cell specialization and combinations thereof.

5. The method according to claim 3 wherein promoter selected is from tissue specific promoter, expresses in plant tissues selected from the group consisting of leaf, stem, root, flower, petal, anther, ovule etc and combinations thereof.

6. The method according to claim 3 wherein promoter selected is from cell type specific promoter, expresses in plant cells selected from the group consisting of parenchyma, mesophyll, xylem, phloem, guard cell, stomatal cell etc and combinations thereof.

7. The method according to claim 2, wherein the glutamate decarboxylase enzyme comprises an amino acid sequence set forth in SEQ ID NO: 2.

8. The method according to claim 7, wherein the amino acid sequence as set forth in SEQ ID No. 2 is effective to catalyze a reaction of glutamic acid to gamma-amino-butyric acid (GABA).

9. The method according to claim 1, wherein the transformed plant expresses glutamate decarboxylase (GAD) gene set forth in SEQ ID No. 1, at higher level than the level of the GAD gene expressed by a non-transformed plant of the same species under the same conditions.

10. The method according to claim 1, wherein the target plant is selected from the group consisting of monocots, dicots, cereals, forage crops, legumes, pulses, vegetables, fruits, oil seeds, fiber crops, flowers, horticultural, medicinal and aromatic plants.

11. The method of claim 1, wherein said incorporating DNA construct into plant genome comprises;
I. Transforming a cell, tissue or organ from a host plant with the DNA construct;
II. Selecting a transformed cell, cell callus, somatic embryo, or seed which contains the DNA construct;
III. Regenerating a whole plant from the selected transformed cell, cell callus, somatic embryo, or seed; and
IV. Selecting a regenerated whole plant that expresses the polynucleotide.

12. The method according to claim 11 wherein a cell tissue or organ from a host plant is transformed with the DNA construct mediated by using particle gun, biolistic or Agrobacterium.

13. A transformed plant obtained according to claims 1-12 and its progeny thereof.

14. The transformed plant according to claim 13, wherein the DNA construct set forth in SEQ No. 1 is incorporated into the plant in a heterozygous or homozygous state.

15. The transformed plant according to claims 1-14, wherein the plant exhibits significantly altered characteristics of plant architecture selected from the group consisting of plant height, internodal distance, stem thickness, number of leaves, leaf size, biomass harvestable yield and combinations thereof.

16. The transformed plant according to claim 15, wherein the plant exhibits significantly enhanced leaf number and or leaf size.

17. The transformed plant according to claim 16, wherein the plant exhibits significantly longer and or broader leaves.

18. The transformed plant according to claim 15, wherein the plant exhibits significantly enhanced biomass.

19. The transformed plant according to claim 15, wherein the plant exhibits significantly enhanced harvestable yield.

20. A plant transformed with a vector comprising a constitutive promoter operably linked to a polynucleotide that encodes a GAD enzyme, or progeny thereof; wherein the plant expresses the polynucleotide; and wherein the plant exhibits significantly improved plant architecture, plant height, internodal distance, stem thickness, number of leaves, leaf size, biomass harvestable yield, reproductive function or other morphological or agronomic characteristic compared to a non-transformed plant.


SEQUENCE LISTING (2 sequences)

SEQ ID NO: 1 - DNA, 1479 bp, Oryza sativa
  CDS (1)..(1479)
atggtgctctccaaggccgtctccgagagtgacatgtccgtgcactccaccttcgcctcccgctacgtccgcgcctccctcccaaggtaccggatgccggagaactcgatcccgaaggaggcggcgtaccagatcatcaacgacgagctgatgctggacggcaacccgcggctgaacctggcgtcgttcgtcaccacgtggatggagcccgagtgcgacaagctcatcatggccgccatcaacaagaactacgtcgacatggacgagtaccccgtcaccaccgagctccagaaccggtgcgtgaacatgatcgcgcacctgttccacgcgccgctcggggaggacgagacggcggtgggcgtgggcacggtgggttcgtcggaggccatcatgctggccgggctggccttcaagcggcggtggcagaacaagcgcaaggccgaggggaagccgttcgacaagcccaacatcatcaccggcgccaacgtgcaggtgtgctgggagaagttcgcccgctacttcgaggtggagctcaaggaggtgaagctccgcgacggctactacgtcatggaccccgagaaggccgtcgacatggtcaacgagaacaccatctgcgtcgccgccatcctcggctccaccctcaacggcgagttcgaggacgtcaagctactcaacgacctcctcgacaagaagaacaaggagactgggtgggagacgccgatccacgtggacgcggcgagcggcgggttcatcgcgccgttcctgtacccggagctggagtgggacttccggctgccgtgggtgaagagcatcaacgtgagcggtcacaagtacgggctcgtctacgccggcatcggctggtgcatctggcgcaacaaggaggacctgcccgaggagctcatcttccacatcaactacctcggcaccgaccagccaaccttcaccctcaacttctccaagggctccagccaggtcatcgcccagtactaccagctcatccgccacggcttcgaggggtacaggaacatcatggagaactgccacgagaacgcgatggtgctgaaggaagggctggtgaagacggggaggttcgacatcgtgtccaaggacgaaggggtgccgctggtggcgttctcgctcaaggaccggagccggcacgacgagttcgagatctccgacatgctgcgccgcttcggctggatcgtgccggcgtacaccatgccgcccgacgcccagcacgtcacggtgctccgcgtggtcatccgggaggagttcagccgcaccctcgccgagcgcctcgtcctcgacatcgagaaggtgatgtaccagctcgacgcgctcccctccaggctcatgccccccgtgccgccggcgccgctgctggtggtcgccaagaagtcggagctcgagacgcagcggtcggtgacggaggcgtggaagaagttcgtgctcgccaagaggaccaacggcgtctgctag

SEQ ID NO: 2 - protein, 492 aa, Oryza sativa
MVLSKAVSESDMSVHSTFASRYVRASLPRYRMPENSIPKEAAYQIINDELMLDGNPRLNLASFVTTWMEPECDKLIMAAINKNYVDMDEYPVTTELQNRCVNMIAHLFHAPLGEDETAVGVGTVGSSEAIMLAGLAFKRRWQNKRKAEGKPFDKPNIITGANVQVCWEKFARYFEVELKEVKLRDGYYVMDPEKAVDMVNENTICVAAILGSTLNGEFEDVKLLNDLLDKKNKETGWETPIHVDAASGGFIAPFLYPELEWDFRLPWVKSINVSGHKYGLVYAGIGWCIWRNKEDLPEELIFHINYLGTDQPTFTLNFSKGSSQVIAQYYQLIRHGFEGYRNIMENCHENAMVLKEGLVKTGRFDIVSKDEGVPLVAFSLKDRSRHDEFEISDMLRRFGWIVPAYTMPPDAQHVTVLRVVIREEFSRTLAERLVLDIEKVMYQLDALPSRLMPPVPPAPLLVVAKKSELETQRSVTEAWKKFVLAKRTNGVC